(19) United States
(12) Patent Application Publication
(10) Pub. No.: US 2025/0041510 A1
(43) Pub. Date: Feb. 6, 2025

(54) INFUSION PUMP MONITORING

(71) Applicant: Welch Allyn, Inc., Skaneateles Falls, NY (US)

(72) Inventors: Tyson B. Whitaker, Arden, NC (US); John A. Lane, Venice, FL (US); WonKyung McSweeney, Manlius, NY (US); David E. Quinn, Moravia, NY (US); Gene J. Wolfe, Pittsford, NY (US)

(21) Appl. No.: 18/787,376

(22) Filed: Jul. 29, 2024

Related U.S. Application Data
(60) Provisional application No. 63/517,773, filed on Aug. 4, 2023.

(51) Int. Cl. A61M 5/142; A61M 5/14; A61M 5/168

(52) U.S. Cl. CPC A61M 5/142; A61M 5/1407; A61M 5/1684; A61M 2205/0227; A61M 2205/10; A61M 2205/18; A61M 2205/3306; A61M 2205/3334; A61M 2205/3386; A61M 2205/3561; A61M 2205/584; A61M 2205/6072; A61M 2209/00

(57) ABSTRACT

A system for monitoring fluid delivery to a patient. The system communicates an alert to a caregiver when a condition that triggers an alarm is confirmed to exist based on video data. The system calculates flow rates of fluids delivered from one or more containers based on monitoring fiducial markings. The system issues an alert when an abnormality is detected based on monitoring a line tracing between an infusion pump and the patient.

### BACKGROUND

Intravenous delivery of drugs and other fluids to patients is a common medical practice. For delivery of larger fluid quantities, typically over 100 mL, these fluids are typically delivered from a container in the form of a sterile bag or bottle. Typically, the container is hung from a pole or rack at a height above a seated or prone patient. Connection between the container and patient is made with an IV administration set, which typically includes a long, flexible polymer tube with a spike fitting at one end and a Luer fitting at the other end. The spike is used to make a sealed, sterile connection to the container. A hypodermic needle is mounted to the Luer fitting to deliver the fluid to the patient's venous system. The IV administration set may also incorporate various other elements including clamps, valves, filters, ports, and the like.

Infusion pumps have been developed to provide accurate and reliable control of the flow of the fluid from the container to the patient. Typically, infusion pumps have a pumping chamber that connects to the disposable IV administration set. The pump mechanism works externally to this sterile pumping chamber to pump a nominally constant volume of fluid during each pumping cycle. The volume pumped per cycle varies from about 0.1 mL to 2 mL per cycle. Modern infusion pumps are computer controlled and typically have user interfaces and programming. These infusion pumps are designed to assist with rate and dosage setting, prevent certain user errors, and detect and respond to certain unsafe conditions during operation.

### SUMMARY

In general terms, the present disclosure relates to infusion pump monitoring. In one possible configuration, an alert is communicated to a caregiver when a condition that triggers an alarm is confirmed to exist based on video data. In another possible configuration, flow rates of fluids delivered from one or more containers are calculated based on monitoring fiducial markings. In another possible configuration, an alert is issued when an abnormality is detected based on monitoring a line tracing between an infusion pump and a patient. Various aspects are described in this disclosure, which include, but are not limited to, the following aspects.

One aspect relates to a system for monitoring fluid delivery to a patient, the system comprising: at least one processing device; and at least one computer readable data storage device storing software instructions that, when executed by the at least one processing device, cause the at least one processing device to: receive an alarm associated with an infusion pump; analyze video data captured by one or more cameras to confirm whether a condition triggering the alarm exists; and send an alert to a communications device associated with a caregiver when the condition that triggers the alarm is confirmed to exist based on the video data.

Another aspect relates to a method of monitoring fluid delivery to a patient, the method comprising: receiving an alarm associated with an infusion pump; analyzing video data captured by one or more cameras to confirm whether a condition triggering the alarm exists; and sending an alert to a communications device when the condition that triggers the alarm is confirmed to exist based on the video data.

Another aspect relates to a system for monitoring fluid delivery to a patient, the system comprising: at least one processing device; and at least one computer readable data storage device storing software instructions that, when executed by the at least one processing device, cause the at least one processing device to: capture video data of one or more containers connected to an infusion pump, each container of the one or more containers holding a fluid for delivery to the patient; calculate flow rates of the fluids from the one or more containers based on monitoring fiducial markings on the one or more containers; determine whether a calculated flow rate of a fluid held in a container matches a flow rate of the fluid set on the infusion pump; and issue an alert when a difference between the calculated flow rate of the fluid and the flow rate of the fluid set on the infusion pump is outside of a predetermined range.

Another aspect relates to a method of monitoring fluid delivery to a patient, the method comprising: capturing video data of one or more containers connected to an infusion pump, each container of the one or more containers holding a fluid for delivery to the patient; calculating flow rates of the fluids from the one or more containers based on monitoring fiducial markings on the one or more containers; determining whether a calculated flow rate of a fluid held in a container matches a flow rate of the fluid set on the infusion pump; and issuing an alert when a difference between the calculated flow rate of the fluid and the flow rate of the fluid set on the infusion pump is outside of a predetermined range.

Another aspect relates to a system for monitoring fluid delivery to a patient, the system comprising: at least one processing device; and at least one computer readable data storage device storing software instructions that, when executed by the at least one processing device, cause the at least one processing device to: identify the patient in video data received from one or more cameras; identify an infusion pump in the video data; perform line tracing from the infusion pump to the patient; detect an abnormality based on monitoring the line tracing between the infusion pump and the patient; and issue an alert when the abnormality is detected.

Another aspect relates to a method of monitoring fluid delivery to a patient, the method comprising: identifying the patient in video data received from one or more cameras; identifying an infusion pump in the video data; performing line tracing from the infusion pump to the patient; detecting an abnormality based on monitoring the line tracing between the infusion pump and the patient; and issuing an alert when the abnormality is detected.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for monitoring fluid delivery to a patient.

FIG. 2 schematically illustrates an example of the system of FIG. 1.

FIG. 3 shows examples of attachment sites for mounting a camera onto an infusion pump of the system of FIG. 1.

FIG. 4 shows an example of the infusion pump of FIG. 3 connected to multiple containers for delivery of multiple fluids to the venous system of the patient.

FIG. 5 schematically illustrates an example of a camera used by the system of FIG. 1.

FIG. 6 shows an example of a container that contains fluid and that can be connected to the infusion pump of FIG. 3 for delivery of the fluid to the patient.

FIG. 7 schematically illustrates an example of a method of monitoring fluid delivery to a patient that can be performed by the system of FIG. 1.

FIG. 8 schematically illustrates another example of a method of monitoring fluid delivery to a patient that can be performed by the system of FIG. 1.

FIG. 9 schematically illustrates another example of a method of monitoring fluid delivery to a patient that can be performed by the system of FIG. 1.

FIG. 10 schematically illustrates an exemplary architecture of a computing device of the system of FIG. 1.

### DETAILED DESCRIPTION

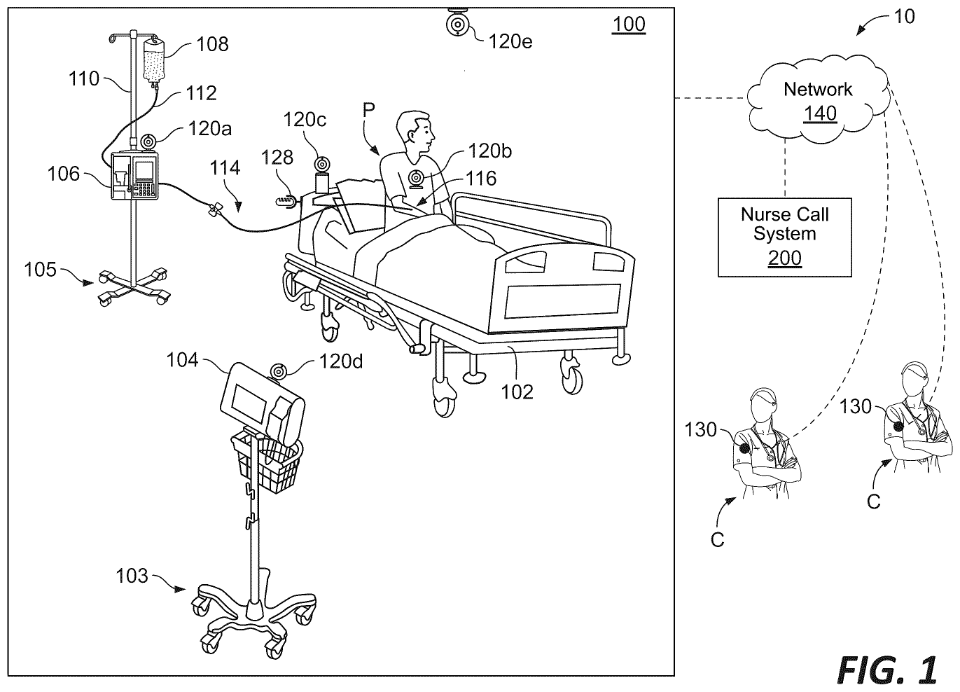

FIG. 1 illustrates an example of a system 10 for monitoring fluid delivery to a patient P. As shown in FIG. 1, the patient P is resting on a patient support apparatus 102 inside a patient environment 100. The patient environment 100 can include an area within a medical facility such as a patient room in a hospital. The patient environment 100 includes medical equipment such as the patient support apparatus 102, as well as a patient monitoring device 104, and an infusion pump 106 that controls intravenous delivery of a fluid from a container 108 to the patient P.

As shown in FIG. 1, the patient P is supported on the patient support apparatus 102 inside the patient environment 100. In some examples, the patient support apparatus 102 is a hospital bed, or similar type of apparatus. The patient support apparatus 102 can include one or more sensors that measure one or more physiological parameters of the patient P such as heart rate, non-invasive blood pressure (NIBP), motion, and weight. Additionally, the patient support apparatus 102 can include sensors that detect patient exit, incontinence, and deterioration.

The patient monitoring device 104 can be used to measure and monitor physiological parameters of the patient P, and to display representations of the measured physiological parameters. The patient monitoring device 104 includes one or more sensor modules that can be used to measure one or more physiological parameters of the patient P. For example, the patient monitoring device 104 can include a temperature sensor module, a photoplethysmogram sensor module, and a non-invasive blood pressure (NIBP) sensor measurement module. As used herein, a “module” is a combination of physical structure which resides in the patient monitoring device 104 and peripheral components that attach to and reside outside of the patient monitoring device 104. The patient monitoring device 104 can include additional sensor modules for receiving additional physiological parameter measurements, including ECG or EKG measurements.

In the example of FIG. 1, the patient monitoring device 104 is mounted on a mobile cart 103 such that the patient monitoring device 104 is portable and can be brought into and out of the patient environment 100. In alternative examples, the patient monitoring device 104 can be stationary such that it can include a wall mounted unit. The patient monitoring device 104 can further include one or more physical assessment devices such as an otoscope, ophthalmoscope, dermatoscope, and other devices for physically assessing the health of the patient P.

In the example of FIG. 1, the infusion pump 106 is mounted on a mobile cart 105 such that the infusion pump 106 is portable and can be brought into and out of the patient environment 100 as needed. The mobile cart 105 includes a pole 110 on which the container 108 hangs. Gravity causes the liquid in the container 108 to flow to the infusion pump 106 via a first tubing 112. Once the liquid reaches the infusion pump 106, the infusion pump 106 controls the flow of the fluid for intravenous delivery of the fluid to a patient attachment site 116 via a second tubing 114. The patient attachment site 116 can include a hypodermic needle that is mounted to a Luer fitting to deliver the fluid to the venous system of the patient P.

As shown in FIG. 1, the system 10 includes a plurality of cameras 120 mounted inside the patient environment 100. For example, a first camera 120a is mounted within or on the infusion pump 106, a second camera 120b is a wearable device that can be worn on the patient P, a third camera 120c is mounted on the patient support apparatus 102, a fourth camera 120d is mounted on the patient monitoring device 104, and a fifth camera 120e is mounted onto a wall or ceiling of the patient environment 100. The system 10 can include additional cameras, or fewer cameras as may be needed for a particular patient environment.

The cameras 120 can each include pan-tilt-zoom movements for adjusting a view of the infusion pump 106 including the container 108, the first and second tubing 112, 114, and the patient attachment site 116. For example, the cameras 120 can each include an electric motor and mechanical pieces that allow the cameras to pan from left to right, tilt up and down, and zoom in and out by adjusting a focal length of a lens. The cameras 120 can monitor expansive open regions that need views in a range of 180 or 360 degrees.

The cameras 120 can be controlled to track movement of the infusion pump 106 and associated components such as the container 108, the first and second tubing 112, 114, and the patient attachment site 116. The cameras 120 can include infrared and/or thermography imaging that can be used to identify the infusion pump 106 including the container 108, the first and second tubing 112, 114, and the patient attachment site 116 in dark and low-light conditions such as at night when the patient P is sleeping on the patient support apparatus 102.

In addition to capturing video data of the infusion pump 106 and its components, the cameras 120 can also be used to capture video data of the patient P. For example, the cameras 120 can be used to capture video data of the patient attachment site 116 such as to monitor whether the second tubing 114 is removed from the patient attachment site 116.

Also, the cameras 120 can be used to capture video data of the patient P that can be used to assess the patient P's interaction with the fluid infusion. For example, the cameras 120 can be used to detect facial expressions of the patient P that can indicate an adverse reaction to the fluid infusion. In further examples, the cameras 120 can be used to monitor patient motion, which can be indicative of a positive or adverse reaction to the fluid infusion.

The video data can also be used to extract vital signs measurements of the patient P to determine whether the patient P is responding to the fluid infusion as intended, or is having an adverse reaction. Examples of the vital signs measurements that can be extracted from the video data can include heart rate, respiration rate, body temperature, and the like. Frequency filters such as infrared and thermography can be used to extract the vital signs measurements from the video data of the patient P. Also, fiducial markings can be attached to the patient P to detect micromovements of the patient P that can be used to determine respiration rate, heart rate, and other physiological parameters of the patient. Several techniques can be used to determine the respiration rate of the patient P from the video data captured by the cameras 120, such as those described in U.S. Provisional Patent Application No. 63/489,901, filed Mar. 13, 2023, entitled Respiration Monitoring, which is incorporated herein by reference in its entirety.

The patient environment 100 can further include a microphone 128 to record sounds inside the patient environment. The microphone 128 can be fixed to a piece of equipment inside the patient environment 100 such as the patient support apparatus 102, the patient monitoring device 104, and/or the infusion pump 106. In the example shown in FIG. 1, the microphone 128 is mounted on the patient support apparatus 102. In some examples, the microphone 128 is part of a pillow speaker included on the patient support apparatus 102. In further examples, the microphone 128 can be fixed to a ceiling or wall of the patient environment 100.

The microphone 128 can detect sounds inside the patient environment 100 such as a caregiver C verbally expressing a change in operation of the infusion pump 106 such as replacement of the container 108, a change in flow rate of the fluid administered to the patient P, or attachment of another container for delivery of another type of fluid to the patient P. Also, the microphone 128 can detect an audible alarm that is triggered on the infusion pump 106. As will be described in more detail, the sounds detected by the microphone 128 inside the patient environment 100 can be utilized by an alarming algorithm to control the operation of the infusion pump 106, and/or to issue alerts or notifications to one or more of the caregivers C.

As further shown in FIG. 1, the equipment in the patient environment 100 is connected to a network 140. For example, the patient support apparatus 102, the patient monitoring device 104, the infusion pump 106, the plurality of cameras 120, and the microphone 128 can all connect to the network 140. In some examples, the network 140 is an Internet of things (IoT) network that connects and exchanges data between the equipment in the patient environment 100, and shares the data with other systems and devices outside of the patient environment 100. For example, as shown in FIG. 1, the network 140 can exchange the data captured by the equipment inside the patient environment 100 with a nurse call system 200 that facilitates communications between the caregivers C in the medical facility. As will be described in more detail, the nurse call system 200 can communicate over the network 140 alarms, alerts, notifications, and the like to communications devices 130 associated with the caregivers C.

The communications devices 130 can include badges that can be worn by the caregivers C that allow the caregivers to receive calls, text messages, and notifications, and that can further allow a caregiver C to broadcast messages to groups of caregivers, initiate and join conference calls, schedule reminders, and the like. The communications devices 130 can also include smartphones, tablet computers, or other types of electronic devices that can be carried by the caregivers C while on shift in the medical facility. In further examples, the communications devices 130 can include one or more computers positioned in a nurses station within the medical facility where the patient environment 100 is located. In further examples, the communications devices 130 can include one or more computers remotely located outside of the medical facility.

The system 10 uses video captured by the plurality of cameras 120 to monitor a status of the fluid delivery to the patient P. Additionally, in some examples, the system 10 can use the sounds recorded by the microphone 128 to control operation of the infusion pump 106. As an illustrative example, the system 10 can use the video captured by the plurality of cameras 120 to confirm or suppress alarms generated by the infusion pump 106. In some examples, the alarms generated by the infusion pump 106 are detected by the microphone 128 when the alarms are generated as audible alarms. Additionally, the system 10 can use the video captured by the plurality of cameras 120 to monitor a status of the infusion pump 106 such as whether a flow rate of the fluid pumped by the infusion pump 106 matches a flow rate set by a clinician C. Also, the system 10 can use the video captured by the plurality of cameras 120 to detect one or more events associated with the intravenous delivery of the fluid to the patient P such as a kinked infusion line, air bubbles, poor port attachment, or empty container.

The system 10 provides remote monitoring and management of the infusion pump 106. By confirming or suppressing the alarms generated by the infusion pump 106, the system 10 can reduce false alarms and alarm fatigue, and reduce the frequency that the caregivers Center the patient environment 100 (e.g., a patient room). This can free up the caregivers C to perform other duties in the medical facility. Also, by monitoring the flow rate of the fluid delivered from the container 108, the system 10 can predict when the container 108 will empty and schedule replacement of the container 108 in advance to optimize the scheduling of tasks for the caregivers C. Such improvements can reduce workloads on the caregivers C, which is especially helpful when the medical facility is experiencing a shortage of caregivers.

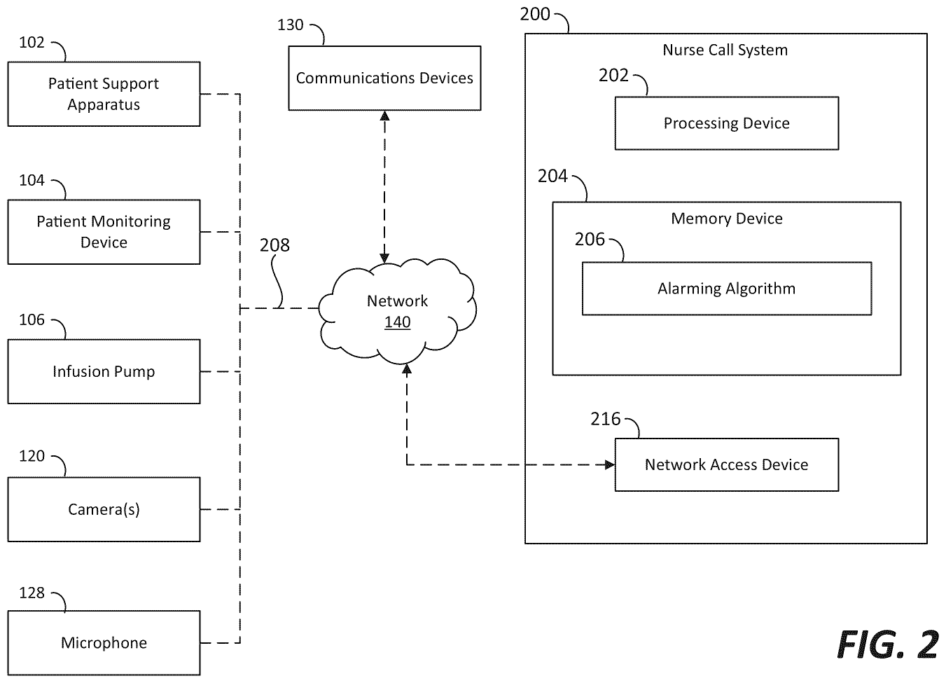

FIG. 2 schematically illustrates an example of the system 10. As shown in FIG. 2, the nurse call system 200 communicates over the network 140 with the patient support apparatus 102, the patient monitoring device 104, the infusion pump 106, the plurality of cameras 120, the microphone 128, and the communications devices 130 worn or otherwise carried by the clinicians C. The network 140 can exchange data between these devices and the nurse call system 200 over the Internet and/or other communications networks.

The nurse call system 200 includes at least one processing device 202. The at least one processing device 202 is an example of a processing unit such as a central processing unit (CPU). The at least one processing device 202 can include one or more central processing units (CPU). In further examples, the at least one processing device 202 includes digital signal processors, field-programmable gate arrays, and similar electronic computing circuits.

The nurse call system 200 includes a memory device 204 that stores data and instructions for execution by the at least one processing device 202. As shown in FIG. 2, the memory device 204 stores an alarming algorithm 206 that uses data received from the equipment in the patient environment 100 to issue an alarm. The alarming algorithm 206 will be described in more detail below. The memory device 204 includes computer readable media, including any media accessible by the nurse call system 200. For example, computer readable media includes computer readable storage media and computer readable communication media.

Computer readable storage media includes volatile and nonvolatile, removable and non-removable media implemented in any device configured to store information such as computer readable instructions, data structures, program modules, or other data. Computer readable storage media can include random access memory, read only memory, electrically erasable programmable read only memory, flash memory, and other memory technology, including any medium that can be used to store information that can be accessed by the nurse call system 200. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The nurse call system 200 includes a network access device 216. The network access device 216 operates to communicate with other devices over the network 140 such as the patient support apparatus 102, the patient monitoring device 104, the infusion pump 106, the plurality of cameras 120, the microphone 128, and the communications devices 130. Examples of the network access device 216 include wired network interfaces and wireless network interfaces.

The network 140 facilitates data communication between the devices inside and outside of the patient environment 100, including the patient support apparatus 102, the patient monitoring device 104, the infusion pump 106, the plurality of cameras 120, the microphone 128, the communications devices 130, and the nurse call system 200. The network 140 includes computing devices and connections therebetween to enable data communication among the computing devices. The network 140 can include routers and other networking devices. The network 140 can include any type of wired or wireless connection, or any combinations thereof. Examples of wireless connections can include cellular network connections such as 4G or 5G, Wi-Fi, Bluetooth, and other similar types of wireless technologies.

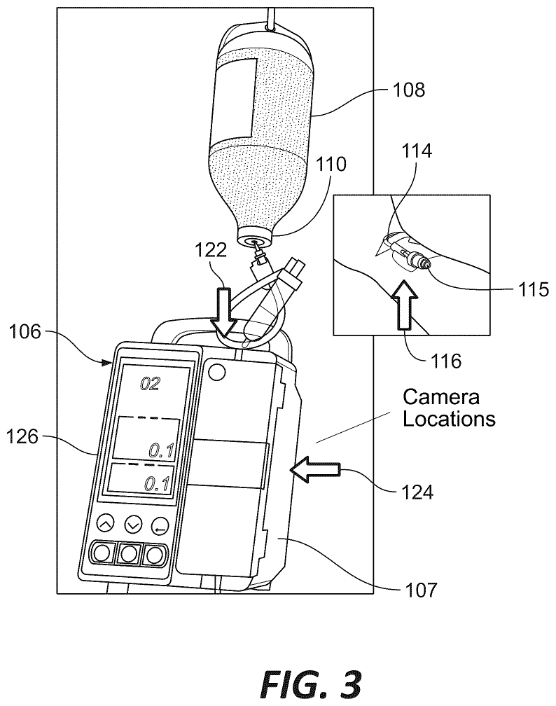

FIG. 3 shows examples of attachment sites for mounting the first camera 120a onto the infusion pump 106. As shown in the examples provided in FIG. 3, the first camera 120a can be mounted on a first attachment site 122 on a top surface of the infusion pump 106. Alternatively, the first camera 120a can be mounted on a second attachment site 124 on a side surface of the infusion pump 106. In both the first and second attachment sites 122, 124, the first camera 120a is mounted on an exterior of the infusion pump 106, and is positioned below one or more containers 108 hung on the pole 110. In such examples, the first camera 120a can be used to simultaneously monitor multiple containers attached to the infusion pump 106.

In further example embodiments, the first camera 120a can be mounted proximate to the patient attachment site 116 where the fluid enters the venous system of the patient P. For example, the first camera 120a can be mounted on a Luer fitting 115 that connects a hypodermic needle to the second tubing 114. In further examples, the system 10 can include cameras 120 mounted to multiple sites associated with the infusion pump 106 such as a camera 120 mounted to the first attachment site 122 and/or a camera 120 mounted to the second attachment site 124 and/or a camera 120 mounted to the Luer fitting 115 next to the patient attachment site 116.

As further shown in FIG. 3, the infusion pump 106 includes an interface 126 mounted on a housing 107. The interface 126 includes controls that are selectable by a caregiver C to adjust the settings for the infusion pump 106 such as a flow rate for delivery of the fluid in the container 108 to the venous system of the patient P. The interface 126 can include a touchscreen and/or one or more physical buttons such as capacitive touch buttons and the like.

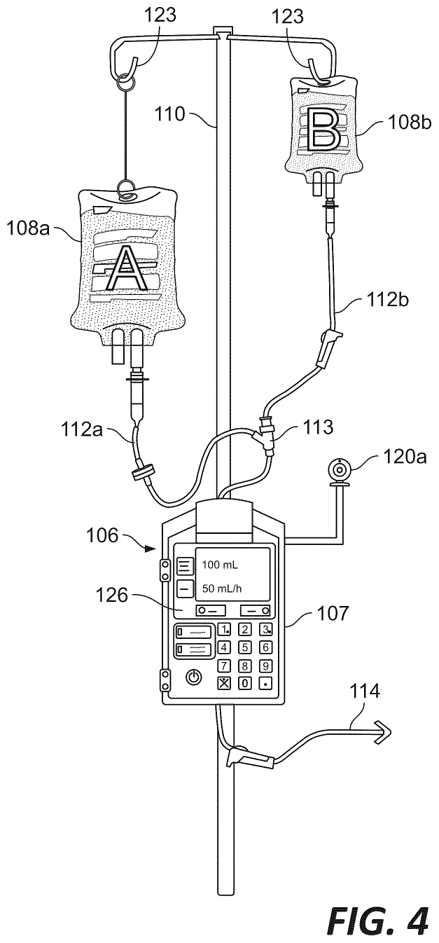

FIG. 4 shows an example of the first camera 120a mounted to the infusion pump 106, which is connected to multiple containers for delivery of multiple fluids to the venous system of the patient P. In this example, a first container 108a is connected to the infusion pump 106 via a first tubing 112a, and a second container 108b is connected to the infusion pump 106 via a first tubing 112b that joins with the first tubing 112a at a connection site 113. As an illustrative example, the first container 108a can include crystalloid solution to treat dehydration of the patient P, while the second container 108b can include a medication such as an antibiotic for treating an infection of the patient P. In further examples, more than two containers can be connected to the infusion pump for delivery of additional fluids to the venous system of the patient P. The data captured by the first camera 120a can be used to monitor the status of the first and second containers 108a, 108b, the infusion pump 106, and the patient attachment site 116.

The infusion pump 106 including the housing 107, the containers 108, the first and second tubing 112, 114, and the Luer fitting 115 can each be labeled for improved identification by the system 10. For example, the housing 107, the containers 108, the first and second tubing 112, 114, and the Luer fitting 115 can each include a coating that emits electromagnetic radiation such as fluorescence when illuminated by a light source. In further examples, the housing 107, the containers 108, the first and second tubing 112, 114, and the Luer fitting 115 can each be impregnated with a dye that emits electromagnetic radiation such as fluorescence when illuminated by a light source. In further examples, the housing 107, the containers 108, the first and second tubing 112, 114, and the Luer fitting 115 can each include a coating of luminescent material, or be impregnated with a dye of luminescent material.

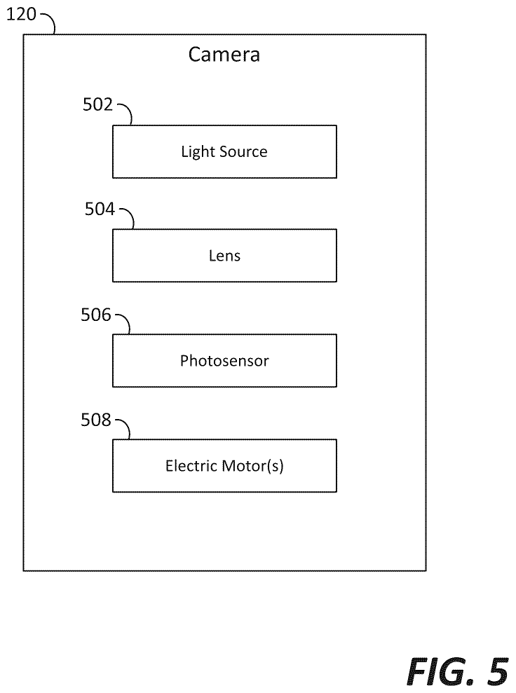

FIG. 5 schematically illustrates an example of a camera 120 that can be used by the system 10. Referring now to FIG. 5, the camera 120 can include a light source 502, a lens 504, and a photosensor 506. The camera 120 can further include one or more electric motors 508 such as stepper motors (also known as step motors or stepping motors), or similar types of electric motors. The one or more electric motors 508 can be used to move one or more components of the camera 120, including the light source 502, the lens 504, and the photosensor 506, such as to pan from left to right, tilt up and down, and zoom in and out.

The light source 502 can include one or more light-emitting diodes (LEDs). The light emitted by the light source 502 is absorbed by objects positioned in front of the camera 120, and the absorption of the light can cause the objects to emit fluorescence. For example, the light source 502 can emit light that causes a coating or dye on the housing 107, the containers 108, the first and second tubing 112, 114, and the Luer fitting 115 to emit fluorescence in the visible light frequency range for improved detection of these components by the system 10. In this manner, the system 10 can more easily identify the infusion pump 106 and the components associated therewith for monitoring a status of the delivery of the fluid by the infusion pump 106.

In further examples, the infusion pump 106 and the components that are associated with the infusion pump 106 such as the housing 107, the containers 108, the first and second tubing 112, 114, and the Luer fitting 115 can each include a unique machine-readable label that includes digital data for identifying the component. For example, quick response (QR) codes or similar types of machine-readable labels can be attached to each of the housing 107, the containers 108, the first and second tubing 112, 114, and the Luer fitting 115 for the system 10 to identify these components connected to the infusion pump 106 in the patient environment 100.

In further examples, the system 10 can perform thermography filtering to distinguish the infusion pump 106 and the components that are associated with the infusion pump 106 from other items that are present inside the patient environment 100. For example, the fluid held in the containers 108 and that is pumped through the first and second tubing 112, 114 can have a temperature that is typically lower than the ambient temperature inside the patient environment 100, and that is lower than the body temperature of the patient P. The system 10 can use the temperature difference to identify the infusion pump 106 and the components associated therewith in the video data captured by the cameras 120 such as by filtering infrared radiation from the video data. In some examples, the cameras 120 can include a thermographic camera (also called an infrared camera or thermal imaging camera, thermal camera, or thermal imager).

In further examples, the system 10 can utilize additional night vision technologies such as image intensification, active illumination in the near infrared or shortwave infrared frequency bands, and other technologies to identify the housing 107, the containers 108, the first and second tubing 112, 114, and the Luer fitting 115 in dark and low-light conditions such as at nighttime when the patient P is sleeping on the patient support apparatus 102.

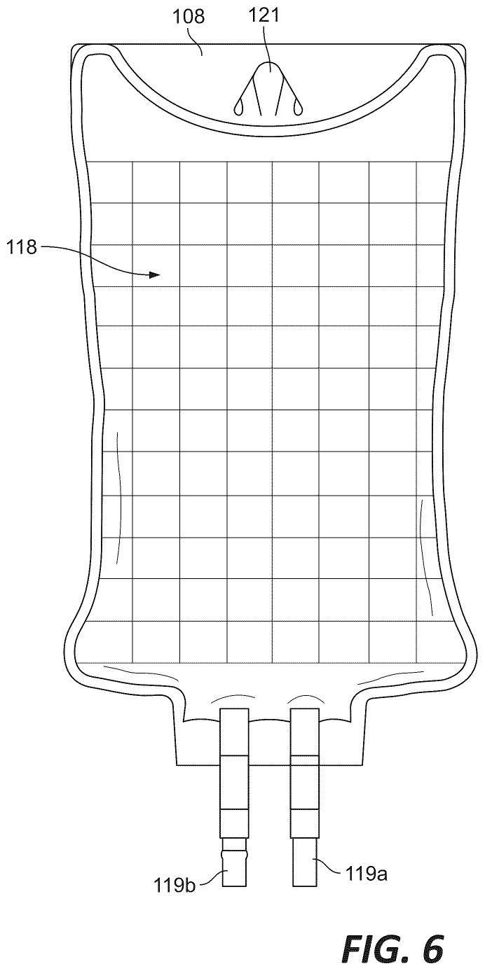

FIG. 6 shows an example of a container 108 that can be connected to the infusion pump 106 for delivery of fluid to the patient P. In this example, the container 108 is a bag that changes shape as the fluid is drained from the bag. For example, the bag is inflated when filled with the fluid, and the bag is deflated when emptied of the fluid. The container 108 includes an aperture 121 for mounting the container 108 onto a hook 123 of the pole 110 (see FIG. 4) and includes one or more ports 119 such as a spiking port 119a for connection to the first tubing 112, and a medication administration port 119b for injecting a medication.

The container 108 includes fiducial markings 118. The system 10 can track gradations in the fiducial markings 118 to monitor a status of the container 108. In further examples, additional components associated with the infusion pump 106, such as the housing 107 and the first and second tubing 112, 114, can include fiducial markings 118 that can be tracked by the system 10 to determine a status of the infusion pump 106.

In the illustrative example shown in FIG. 6, the fiducial markings 118 on the container 108 include a grid pattern. The system 10 can track the grid pattern over time to monitor a status of the container 108. For example, the grid pattern will gradually deform as a result of the container 108 being emptied of the fluid. The system 10 can track deformation of the grid pattern to determine a status of the container 108 such as whether the container 108 is full, partially full, or empty. Also, the system 10 can track deformation of the grid pattern to determine a flow rate of the fluid pumped to the patient P. For example, the system 10 can determine a speed at which the grid pattern deforms to determine the flow rate of the fluid (i.e., a faster deformation of the grid pattern indicates a faster flow rate; a slower deformation of the grid pattern indicates a slower flow rate).

While a grid pattern is shown in the example provided in FIG. 6, the fiducial markings 118 can have other types of patterns and shapes that can be similarly tracked for monitoring a status of the container 108 including whether the container is full, partially full, or empty, and to determine a flow rate of the fluid delivered from the container 108 to the patient P.

In further examples, the fiducial markings 118 can include a color that transitions to a different color as the container 108 is emptied. For example, the container 108 can have a first color when full of the fluid that gradually transitions to a second color as a result of the container 108 being emptied of the fluid. The system 10 can track the color transition to determine a status of the container 108 such as whether the container 108 is full, partially full, or empty.

The system 10 can also track the color transition to determine a flow rate of the fluid pumped to the patient P. For example, the system 10 can determine a speed at which the first color transitions into the second color to determine the flow rate of the fluid (i.e., a faster color transition indicates a faster flow rate; a slower color transition indicates a slower flow rate). Additional examples of using the fiducial markings 118 to determine the flow rate of the fluid from the container 108 are possible.

In further examples, the fiducial markings 118 can be used by the system 10 to detect vibrations of the housing 107, the container 108, the first tubing 112, and/or the second tubing 114 that are indicative of the fluid being pumped by the infusion pump 106. For example, a pattern, a machine-readable label, or other type of fiducial marking can be attached to these components to help visualize minute vibrations that correlate to the pumping action of the infusion pump 106. The system 10 can monitor these vibrations over time to determine a flow rate of the fluid delivery to the patient P, and can thereafter compare the determined flow rate to a flow rate set by a caregiver C to confirm whether the infusion pump 106 is operating correctly.

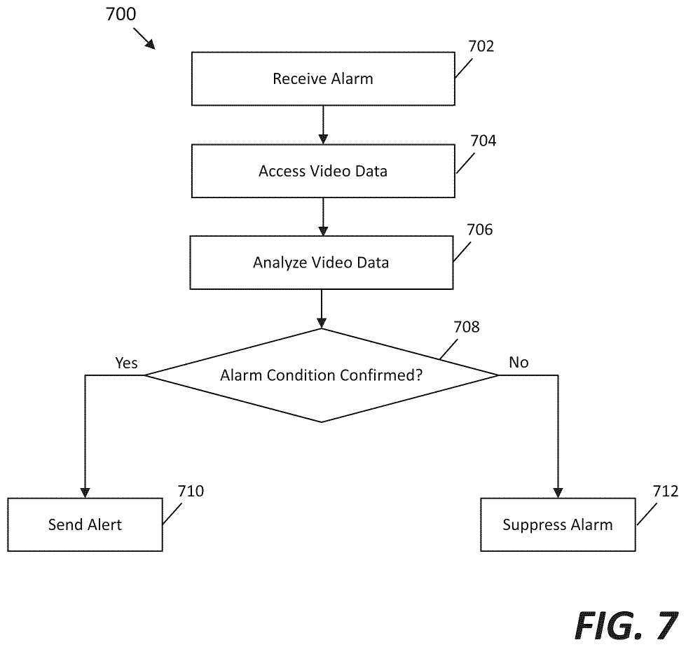

FIG. 7 schematically illustrates an example of a method 700 of monitoring fluid delivery to the patient P. The method 700 can be performed by the nurse call system 200, which is connected via the network 140 to the equipment in the patient environment 100 and to the communications devices 130 of the caregivers C. In some examples, the method 700 is part of the alarming algorithm 206 stored on the memory device 204 of the nurse call system 200.

As shown in FIG. 7, the method 700 includes an operation 702 of receiving an alarm associated with the infusion pump 106. In some examples, the alarm is triggered on the infusion pump 106 when the infusion pump detects a condition associated with the delivery of the fluid to the patient P. In some examples, the alarm is communicated by the infusion pump 106 to the nurse call system 200 via the network 140. In other examples, the nurse call system 200 can detect the alarm such as based on video data captured by the cameras 120 inside the patient environment showing a visible alarm triggered on the infusion pump 106, and/or when the microphone 128 detects an audible alarm triggered on the infusion pump 106.

The alarm can be triggered on the infusion pump 106 when the infusion pump detects a condition affecting delivery of fluid to the patient P. For example, the alarm can be triggered when the infusion pump 106 detects the container 108 is empty, or the fluid flow is interrupted due to a blockage in the first tubing 112 and/or the second tubing 114, presence of bubbles in the first tubing 112 and/or the second tubing 114, detachment of the first tubing 112 from the container 108 or from the infusion pump 106, detachment of the second tubing 114 from the infusion pump 106 or from the patient attachment site 116, a kink in the first and second tubing 112, 114, and other conditions that can inhibit the delivery of the fluid to the patient P.

The method 700 includes an operation 704 of accessing video data captured by one or more of the cameras 120 in the patient environment 100. Operation 704 can include accessing video data from one or more of the first camera 120a mounted within or on the infusion pump 106, the second camera 120b worn on the patient P, the third camera 120c mounted on the patient support apparatus 102, the fourth camera 120d mounted on the patient monitoring device 104, and the fifth camera 120e mounted onto a wall or ceiling of the patient environment 100. The nurse call system 200 can access the video data from the one or more cameras 120 included in the patient environment 100 via the network 140. In this manner, multiple streams of video data can be accessed from the cameras 120, which can each have a different viewpoint of the infusion pump 106 and associated components in the patient environment 100.

The method 700 includes an operation 706 of analyzing the video data accessed in operation 704. Operation 706 can include image processing to identify the infusion pump 106 and associated components such as the housing 107, the container 108, the first and second tubing 112, 114, and/or the Luer fitting 115. In some examples, the image processing includes using machine learning to identify the infusion pump 106 and associated components such as the housing 107, the container 108, the first and second tubing 112, 114, and/or the Luer fitting 115.

Operation 706 can include detecting fluorescence that results from absorption of light by a coating or dye applied on the housing 107, the container 108, the first and second tubing 112, 114, and/or the Luer fitting 115. As a further example, operation 706 can include detecting luminescence by a coating or dye applied on the housing 107, the container 108, the first and second tubing 112, 114, and/or the Luer fitting 115. As a further example, operation 706 can include detecting machine-readable labels such as quick response (QR) codes that are attached to the housing 107, the containers 108, the first and second tubing 112, 114, and/or the Luer fitting 115. As a further example, operation 706 can include applying frequency filtering such as infrared or thermography filtering to identify the infusion pump 106 and the components attached to the infusion pump in the video data captured by the one or more cameras 120.

In further examples, operation 706 can include identifying one or more fiducial markings 118 for determining a status of one or more components associated with the infusion pump 106. For example, operation 706 can include using the fiducial markings 118 in accordance with the examples described above to determine whether one or more containers 108 connected to the infusion pump 106 are empty. As another example, operation 706 can use the fiducial markings 118 to determine whether the first tubing 112 is detached from the container(s) 108 and/or from the infusion pump 106, and/or whether second tubing 114 is detached from infusion pump 106 and/or from the patient attachment site 116. Operation 706 can further include analyzing the video data to determine whether a blockage is present in the first tubing 112 and/or the second tubing 114, whether bubbles are present in the first tubing 112 and/or the second tubing 114, and/or whether the first and second tubing 112, 114 are kinked.

The method 700 includes an operation 708 of confirming the alarm received in operation 702 based on the analysis of the video data in operation 706. Operation 708 can include confirming the condition that triggered the alarm. For example, operation 708 can include confirming whether the container 108 is empty or not, or confirming whether delivery of the fluid in the container 108 to the patient P is interrupted due to a blockage in the first tubing 112 and/or the second tubing 114, bubbles in the first tubing 112 and/or the second tubing 114, detachment of the first tubing 112 from the container 108 and/or from the infusion pump 106, detachment of the second tubing 114 from the infusion pump 106 and/or from the patient attachment site 116, and other conditions that can inhibit the delivery of the fluid to the patient P.

When the condition that triggered the alarm is confirmed (i.e., “Yes” in operation 708), the method 700 proceeds to an operation 710 of sending an alert to a communications device 130. The alert can be generated by the nurse call system 200. The alert can be sent by the nurse call system 200 to the communications device 130 over the network 140. The alert can include an identification of the condition that triggered the alarm such that the caregiver C can be prepared to fix the condition before entering the patient environment. For example, when operation 708 confirms that the container 108 is empty, the alert can include a request to replace the container 108 such that the caregiver C can retrieve a replacement container before entering the patient environment 100. This can reduce the number of visits the caregiver makes to the patient environment 100, and thereby free up the caregiver to perform other tasks.

Otherwise, when the condition that triggered the alarm is not confirmed (i.e., “No” in operation 708), the method 700 proceeds to an operation 712 of suppressing the alarm on the infusion pump 106. In some examples, the nurse call system 200 can communicate with the infusion pump 106 via the network 140. This reduces false alarms and alarm fatigue because alerts are sent to the communications device 130 of the caregiver C only when a condition that triggers an alarm on the infusion pump 106 is confirmed by the nurse call system 200.

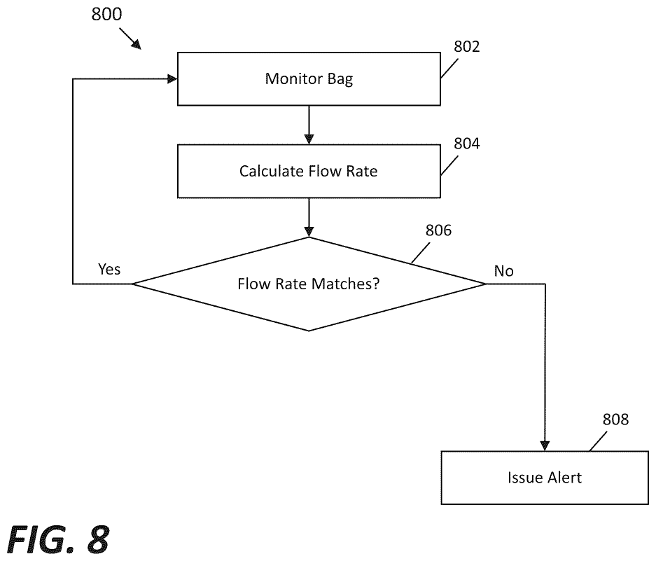

FIG. 8 schematically illustrates another example of a method 800 of monitoring fluid delivery to the patient P. In some examples, the method 800 can be performed by the nurse call system 200, which is connected via the network 140 to the equipment in the patient environment 100 and to the communications devices 130 carried by the caregivers C. In some examples, the method 800 is part of the alarming algorithm 206 stored on the memory device 204 of the nurse call system 200. In alternative examples, the method 800 can be performed by the infusion pump 106, or by another piece of equipment inside the patient environment 100.

As shown in FIG. 8, the method 800 includes an operation 802 of monitoring one or more containers 108 connected to the infusion pump 106. Operation 802 can include monitoring the one or more containers 108 based on video data received from the first camera 120a mounted on the infusion pump 106. Alternatively, or additionally, operation 802 can include monitoring the one or more containers 108 based on video data received from the second camera 120b mounted on the patient P. Alternatively, or additionally, operation 802 can include monitoring the one or more containers 108 based on video data received from the third camera 120c mounted on the patient support apparatus 102, video data received from the fourth camera 120d mounted on the patient monitoring device 104, and/or video data received from the fifth camera 120e mounted onto a wall or ceiling of the patient environment 100. The video data from the one or more cameras 120 in the patient environment 100 can be received via the network 140.

Next, the method 800 includes an operation 804 of calculating a flow rate of the fluid from the one or more containers 108. Operation 804 can include using the fiducial markings 118 on the containers 108 to determine the flow rate of the fluid. For example, operation 804 can include detecting a rate of change in the fiducial markings 118 to determine the flow rate.

As an example, when the fiducial markings include a grid pattern or other type of pattern or design, operation 804 can include detecting a rate that the pattern deforms as the container 108 deflates from the fluid being emptied from the container. The rate of deformation of the fiducial markings 118 can be used to determine a flow rate of the fluid from the container 108. For example, a faster deformation of the fiducial markings 118 indicates a faster flow rate, and a slower deformation of the fiducial markings 118 indicates a slower flow rate.

As another illustrative example, when the fiducial markings 118 include a color that transitions into a different color as the container 108 deflates from the fluid being emptied from the container 108, operation 804 can include detecting a rate of color change to determine a flow rate of the fluid. A faster color transition of the fiducial markings 118 indicates a faster flow rate, and a slower color transition of the fiducial markings 118 indicates a slower flow rate.

Next, the method 800 includes an operation 806 of determining whether the flow rate calculated in operation 804 matches a flow rate set on the infusion pump 106. The flow rate set on the infusion pump 106 is based on an input entered on the interface 126 by a caregiver C or other trained medical professional. For example, the caregiver C or other trained medical professional can enter the flow rate using a touchscreen and/or one or more physical buttons such as capacitive touch buttons on the interface 126 of the infusion pump 106. In some instances, the flow rate set on the infusion pump 106 is determined from the sounds recorded by the microphone 128 inside the patient environment 100 such as when a caregiver C verbally expresses a flow rate for delivery of the fluid by the infusion pump 106 to the patient P.

Operation 804 can include determining whether the flow rate calculated in operation 804 is within a predetermined margin of error of the flow rate set on the infusion pump 106. This can allow for some tolerance between the flow rate calculated in operation 804 and the flow rate set on the infusion pump 106 such that there does not need to be an exact match, but rather there can be an acceptable tolerance between the two flow rates. For example, operation 804 can include determining whether the flow rate calculated in operation 804 is within a 5% margin of error of the flow rate set on the infusion pump 106 to determine whether there is match or not.

The difference between the flow rate calculated in operation 804 and the flow rate set on the infusion pump 106 can be caused by one or more conditions on the infusion pump. For example, the flow rates can differ because the container 108 is empty such that the delivery of the fluid to the patient P has stopped. Also, the flow rates can differ due to an interruption such as a blockage in the first tubing 112 and/or the second tubing 114, bubbles in the first tubing 112 and/or the second tubing 114, detachment of the first tubing 112 from the container 108 or from the infusion pump 106, detachment of the second tubing 114 from the infusion pump 106 or from the patient attachment site 116, a kink in the first and second tubing 112, 114, and other conditions that can inhibit the delivery of the fluid to the patient P.

When the flow rate calculated in operation 804 matches the flow rate set on the infusion pump 106 (i.e., “Yes” in operation 806), the method 800 can return to operation 802 and continue to monitor the one or more containers 108 connected to the infusion pump 106.

Otherwise, when the flow rate calculated in operation 804 does not match the flow rate set on the infusion pump 106 (i.e., “No” in operation 806), such as when a difference between the flow rate calculated in operation 804 and the flow rate set on the infusion pump 106 is outside of a predetermined margin of error, the method 800 proceeds to an operation 808 of issuing an alert. In some instances, the alert is generated as a visual or audible alarm generated on the infusion pump 106. Alternatively, or additionally, the alert can be sent over the network 140 to a communications device 130 carried by a caregiver C. In this manner, the method 800 can be performed to remotely monitor the operation of the infusion pump 106. This can reduce the frequency that the caregivers C enter the patient environment 100 to check on the status of the infusion pump 106, and thereby reduce the workload of the caregivers. This can also improve patient care by ensuring that the infusion pump 106 is properly operating as intended.

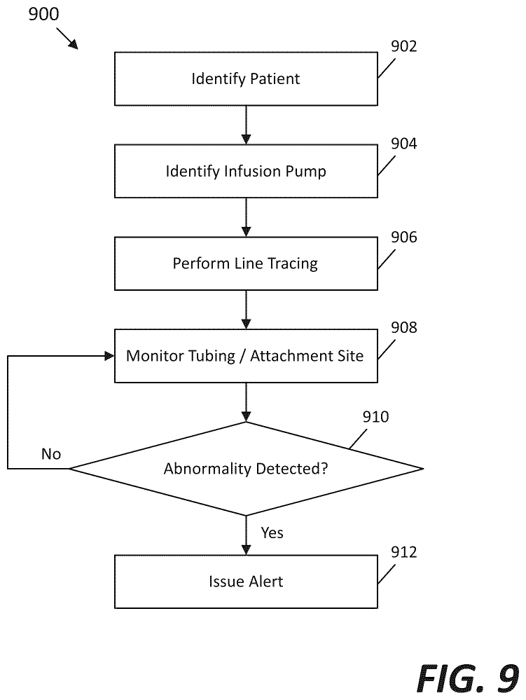

FIG. 9 schematically illustrates another example of a method 900 of monitoring fluid delivery to the patient P. In some examples, the method 900 can be performed by the nurse call system 200, which is connected via the network 140 to the equipment in the patient environment 100 and to the communications devices 130 carried by the caregivers C. In some examples, the method 900 is part of the alarming algorithm 206 stored on the memory device 204 of the nurse call system 200. In alternative examples, the method 900 can be performed by the infusion pump 106, or by another piece of equipment inside the patient environment 100.

As shown in FIG. 9, the method 900 includes an operation 902 of identifying the patient P in video data received from one or more of the cameras 120 inside the patient environment 100. Operation 902 can include identifying the patient P by using infrared or thermography filtering that use the patient P's body temperature to distinguish the patient P from other objects in the patient environment. In other examples, operation 902 can include identifying the patient P by using facial recognition or similar types of technologies. In some examples, operation 902 includes using artificial intelligence to identify and distinguish the patient P from other objects in the patient environment 100.

The method 900 includes an operation 904 of identifying the infusion pump 106. Operation 904 can include identifying the infusion pump 106 by using infrared or thermography filtering to distinguish the infusion pump 106 from other objects in the patient environment. In some examples, operation 904 includes using artificial intelligence to identify and distinguish the infusion pump 106 from other objects in the patient environment 100.

Next, the method 900 includes an operation 906 of performing line tracing from the infusion pump 106 identified in operation 904 to the patient P identified in operation 902. The line tracing is performed to approximate the connection of the second tubing 114 to the patient attachment site 116 (see FIG. 1) for delivery of the fluid to the venous system of the patient P.

Next, the method 900 includes an operation 908 of monitoring the line tracing drawn between the infusion pump 106 and the patient P in the patient environment 100. As an illustrative example, operation 908 can include monitoring a distance between the infusion pump 106 and the patient P. As another illustrative example, operation 908 can include monitoring for a presence of objects positioned between the infusion pump 106 and the patient P.

Next, the method 900 includes an operation 910 of determining whether an abnormality is detected. As an illustrative example, operation 910 can include detecting an abnormality when the infusion pump 106 and the patient P are separated by a distance that suggests the patient P is no longer connected to the infusion pump 106 such as when the distance exceeds an acceptable threshold. As another illustrative example, operation 910 can include detecting an abnormality when an objected is positioned between the infusion pump 106 and the patient P. In such scenarios, the object can interfere with the connection of the second tubing 114 to the patient attachment site 116 on the patient P, or cause a kink in the second tubing 114 that can obstruct the flow of the fluid from the infusion pump 106 to the patient P.

When an abnormality is not detected (i.e., “No” in operation 910), the method 900 can return to monitoring the line tracing drawn between the infusion pump 106 and the patient P in the patient environment 100. When an abnormality is detected (i.e., “Yes” in operation 910), the method 900 can proceed to an operation 912 of issuing an alert.

In some instances, the alert is generated as a visual or audible alarm generated on the infusion pump 106. Alternatively, or additionally, the alert can be sent over the network 140 to a communications device 130 carried by a caregiver C. In this manner, the method 900 can be performed to remotely monitor the operation of the infusion pump 106. This can reduce the frequency that the caregivers C enter the patient environment 100 to check on the status of the infusion pump 106, and thereby reduce the workload of the caregivers. This can also improve patient care by ensuring that the infusion pump 106 is properly operating as intended.

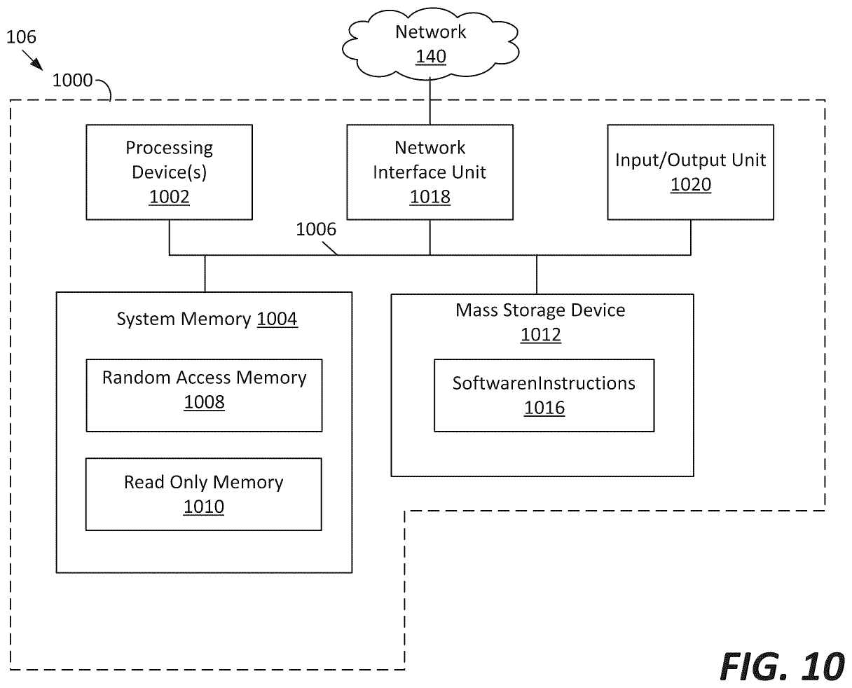

FIG. 10 schematically illustrates an exemplary architecture of a computing device 1000 of the system 10. The computing device 1000 is used to execute the functionality of the system 10 described herein. For example, the infusion pump 106 can include all or some of the elements described with reference to FIG. 10, with or without additional elements. Also, the other equipment in the patient environment 100 such as the patient support apparatus 102 and the patient monitoring device 104 can include similar types of computing devices.

The computing device 1000 includes at least one processing device 1002. Examples of the at least one processing device 1002 can include central processing units (CPUs), digital signal processors, field-programmable gate arrays, and other types of electronic computing circuits. The at least one processing device 1002 can be part of a processing circuitry having a memory for storing instructions which, when executed by the processing circuitry, cause the processing circuitry to perform the functionalities described herein.

The computing device 1000 also includes a system memory 1004, and a system bus 1006 that couples various system components including the system memory 1004 to the at least one processing device 1002. The system bus 1006 can include any type of bus structure including a memory bus, or memory controller, a peripheral bus, and a local bus.

The system memory 1004 can include a random-access memory (“RAM”) 1008 and a read-only memory (“ROM”) 1010. The ROM 1010 provides non-volatile storage of computer-readable instructions (including application programs), data structures, and other data.

The computing device 1000 can also include a mass storage device 1012 that includes software instructions 1016. The mass storage device 1012 is connected to the at least one processing device 1002 through the system bus 1006. The mass storage device 1012 and associated computer-readable data storage media provide non-volatile, non-transitory storage of the software instructions 1016. It should be appreciated by those skilled in the art that computer-readable data storage media can be any available non-transitory, physical device or article of manufacture from which the computing device 1000 can read data and/or instructions. The computer-readable storage media can be comprised of entirely non-transitory media. The mass storage device 1012 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The ROM 1010 and/or the mass storage device 1012 can store software instructions and data. The software instructions can include the alarming algorithm 206, which when executed by the at least one processing device 1002, cause the at least one processing device 1002 to provide the functionality of the system 10 described herein.

The computing device 1000 operates in a networked environment using logical connections to the other devices and systems through the network 140. The computing device 1000 connects to the network 140 through a network interface unit 1018 connected to the system bus 1006. The network interface unit 1018 can connect to additional types of communications networks and devices, including through Bluetooth, Wi-Fi, and cellular telecommunications.

The computing device 1000 includes an input/output unit 1020 for receiving and processing inputs and outputs. Examples of the input/output unit 1020 can include the interface 126 (see also FIGS. 3 and 4). As described above, the interface 126 includes controls that are selectable to adjust the settings for the infusion pump 106 such as a flow rate for delivery of the fluid in the container 108 to the venous system of the patient P. The interface 126 can include a touchscreen and/or one or more physical buttons such as capacitive touch buttons and the like.

The various embodiments described above are provided by way of illustration only and should not be construed to be limiting in any way. Various modifications can be made to the embodiments described above without departing from the true spirit and scope of the disclosure.

What is claimed is:

1. A system for monitoring fluid delivery to a patient, the system comprising:
at least one processing device; and
at least one computer readable data storage device storing software instructions that, when executed by the at least one processing device, cause the at least one processing device to:
receive an alarm associated with an infusion pump;
analyze video data captured by one or more cameras to confirm whether a condition triggering the alarm exists; and
send an alert to a communications device associated with a caregiver when the condition that triggers the alarm is confirmed to exist based on the video data.

2. The system of claim 1, wherein the instructions, when executed by the at least one processing device, further cause the at least one processing device to:
suppress the alarm on the infusion pump when the condition that triggers the alarm is not confirmed to exist based on the video data.

3. The system of claim 1, further comprising:
the one or more cameras, wherein each camera of the one or more cameras is configured to pan from side to side, tilt up and down, and zoom in and out.

4. The system of claim 3, wherein the one or more cameras include at least one of a first camera mounted on the infusion pump, a second camera worn on the patient, a third camera mounted on a patient support apparatus, a fourth camera mounted on a patient monitoring device, and a fifth camera mounted onto a wall or ceiling of an environment where the infusion pump is placed.

5. The system of claim 1, further comprising:
a component connected to the infusion pump, the component including a coating of luminescent material or being impregnated with a dye of luminescent material.

6. The system of claim 1, further comprising:
a component connected to the infusion pump, the component including a coating that fluoresces when illuminated by a light source.

7. The system of claim 1, further comprising:
a component connected to the infusion pump, the component being impregnated with a dye that fluoresces when illuminated by a light source.

8. The system of claim 1, further comprising:
a component connected to the infusion pump, the component including a machine-readable label that includes digital data identifying the component.

9. The system of claim 1, wherein the instructions, when executed by the at least one processing device, further cause the at least one processing device to:
apply frequency filtering to identify the infusion pump and one or more components connected to the infusion pump in the video data.

10. The system of claim 1, wherein the instructions, when executed by the at least one processing device, further cause the at least one processing device to:
use machine learning to identify the infusion pump and one or more components connected to the infusion pump in the video data.

11. A method of monitoring fluid delivery to a patient, the method comprising:
receiving an alarm associated with an infusion pump;
analyzing video data captured by one or more cameras to confirm whether a condition triggering the alarm exists; and
sending an alert to a communications device when the condition that triggers the alarm is confirmed to exist based on the video data.

12. The method of claim 11, further comprising:
suppressing the alarm on the infusion pump when the condition that triggers the alarm is not confirmed to exist based on the video data.

13. The method of claim 11, further comprising:
controlling at least one camera of the one or more cameras to pan from side to side, tilt up and down, and zoom in and out to adjust a view of the video data.

14. The method of claim 11, further comprising:
detecting luminescence to identify the infusion pump and one or more components connected to the infusion pump in the video data.

15. A system for monitoring fluid delivery to a patient, the system comprising:
at least one processing device; and
at least one computer readable data storage device storing software instructions that, when executed by the at least one processing device, cause the at least one processing device to:
capture video data of one or more containers connected to an infusion pump, each container of the one or more containers holding a fluid for delivery to the patient;
calculate flow rates of the fluids from the one or more containers based on monitoring fiducial markings on the one or more containers;
determine whether a calculated flow rate of a fluid held in a container matches a flow rate of the fluid set on the infusion pump; and
issue an alert when a difference between the calculated flow rate of the fluid and the flow rate of the fluid set on the infusion pump is outside of a predetermined range.

16. The system of claim 15, wherein calculate the flow rates of the fluids from the one or more containers includes detecting a rate of change in the fiducial markings.

17. The system of claim 15, wherein the fiducial markings include patterns that deform as the one or more containers empty of the fluids.

18. The system of claim 15, wherein the fiducial markings include a first color that transitions into a second color as the one or more containers empty of the fluids.

19. The system of claim 15, wherein the alert is generated on the infusion pump.

20. The system of claim 15, wherein the alert is sent over a network to a communications device associated with a caregiver.
